(19) United States
(12) Patent Application Publication
(10) Pub. No.: US 2012/0244521 A1
(43) Pub. Date: Sep. 27, 2012

(54) Methods for identifying a virulent strain of a virus

(76) Inventor: Gennadi V. Glinsky, LaJolla, CA (US)

(21) Appl. No.: 13/261,189

(22) PCT Filed: Sep. 2, 2010
(86) PCT No.: PCT/US10/47667

§ 371 Date: Feb. 23, 2012

Related U.S. Application Data
(60) Provisional application No. 61/239,624, filed on Sep. 3, 2009.
(60) Provisional application No. 61/253,379, filed on Oct. 20, 2009.

(51) Int. Cl. C12Q 1/70; G06F 19/10; G01N 33/569

(52) U.S. Cl. 435/5; 702/19

(57) ABSTRACT

The present invention relates to methods for identifying a virulent strain of a virus, particularly and influenza virus, by detecting specific mutations in the amino acid sequence of the hemagglutinin (HA) protein and by determining the case fatality rate for hospitalization (CFR/H) as the number of persons hospitalized for infection by the virus who die from the infection compared to the total number of persons hospitalized for infection, wherein the identification of mutations in the HA protean and/or an increasing CFR/H over time indicates a virulent strain of the virus.

### CROSS REFERENCE TO RELATED APPLICATIONS

This application claims priority to U.S. Provisional Application Nos. 61/239,624 filed Sep. 3, 2009 and 61/253,379, filed Oct. 20, 2009, each of which is incorporated herein by reference in its entirety.

### INCORPORATION BY REFERENCE OF SEQUENCE LISTING

The content of the text file named “26141_{—}512001WO-Seq_List_ST25.txt” which was created on Sep. 1, 2010, and is 10,798 bytes in size, is hereby incorporated by reference in its entirety.

### FIELD OF THE INVENTION

The invention relates to compositions and methods for the identification of a virulent strain of a virus, preferably an influenza virus.

### BACKGROUND OF THE INVENTION

The interconnectedness of human populations in the modern world has contributed to the ability of infectious diseases to spread more rapidly than at any other time in human history. Consequently, the management of outbreaks, and the prevention or at least containment of pandemics, requires a coordinated global effort. This effort includes monitoring and surveillance activities to identify an outbreak in its earliest stages as well as a coordinated public health response to contain and treat an outbreak when it occurs. During an outbreak, it is important to rapidly characterize the infectious vector to provide healthcare workers with the tools they need to assess and respond effectively.

A pandemic is an infectious disease that spreads through a population across a large geographical region, such as one or more continents. Examples of recent pandemics include the Spanish flu pandemic of 1918-1919, the Asian flu pandemic of 1956-1958, the Hong Kong flu pandemic of 1968-1969, and most recently the swine flu pandemic of 2009-2010.

The severity of a pandemic is often measured in terms of a case fatality rate (CFR). The CFR is the proportion of deaths in the population of infected individuals. For example, 9 deaths per 10,000 people formally diagnosed with a disease within a given year would give a CFR of 0.09%. The Spanish flu pandemic, which is estimated to have killed about 20-100 million people worldwide, had a CFR of more than 2.5%. In comparison, more recent pandemics have had much lower CFRs. The Asian and Hong Kong flus, which nevertheless killed about 1-2 million people, each had a CFR of less than 0.1%. The most recent pandemic, the so-called “swine flu” of 2009-2010 is estimated to have had a CFR of about 0.03%. At least some of the credit for the lower CFRs during more recent flu pandemics can be attributed to improvements in healthcare and access to healthcare, as well as increased global preparedness in the form of monitoring and surveillance programs, vaccination programs, and clinical disease management.

The swine flu pandemic of 2009-2010 was caused by the H1N1 strain of influenza A, which is the same serotype as the virus that caused the Spanish flu pandemic of 1918. Although the recent swine flu pandemic was relatively mild in terms of fatalities, there remains a concern among public health officials that a more virulent form of this virus will emerge as it mixes with other viruses in humans, pigs, and birds. In June 2010 the discovery of a hybrid of the H1N1 swine flu virus in pigs underscored this concern (Vijaykrishna, D. et al., Science (2010) 328:1529). The mixing of the genetic material among two viruses that are infecting the same cell is called “reassortment.” The genome of the influenza viruses consists of eight RNA segments that act like mini-chromosomes. These segments can be swapped between different viruses in the same cell, generating novel reassortment strains. Reassortment strains are often the source of pandemics, including the Asian and Hong Kong pandemic flu strains which were reassortments between an avian virus and a human virus. The H1N1 swine flu virus of 2009 was also a reassortment strain. It contained an unusual mix of swine, avian and human influenza genetic sequences. In particular, the hemagglutinin (HA), neuraminidase (NA), nucleoprotein (NP), matrix protein (M), and non-structural proteins (NS/NEP) sequences were of swine origin while the RNA polymerase subunits PA and PB2 were of avian origin and the RNA polymerase subunit PB 1 was of human origin.

Methods are needed to rapidly assess the virulence of a virus during an outbreak and to monitor the evolution of the virus during an ongoing outbreak or pandemic. Early identification of strains having increased virulence will enable public health officials to effectively manage the response to an outbreak or pandemic.

### SUMMARY OF THE INVENTION

The present invention provides methods for identifying a virulent strain of a virus in a population, preferably a human population. In one embodiment, the method comprises detecting a mutation within amino acid residues 301 to 316 of the hemagglutinin protein (HA) of the virus, wherein the virus was isolated from human cells or tissues, and wherein the detection of one or more mutations indicates that a virulent strain of the virus has been identified. In one embodiment, the virus is an influenza virus. In a preferred embodiment, the virus is an influenza A virus. In a specific embodiment, the virus is of the H1 serotype or subtype. The terms serotype and subtype are used interchangeably in this context. In one embodiment, the mutation is with reference to the amino acid sequence GAINTSLPFQNIHPIT (SEQ ID NO:5).

In one embodiment, the mutation is selected from one of the following: a substitution of glutamine at amino acid position 310 with histidine (Q310H); the triple substitution of threonine at position 305 with serine, isoleucine at position 312 with valine, and isoleucine at position 315 with valine (T305S+I312V+I315V); a substitution of isoleucine at position 315 with valine (I315V); a substitution of proline at 314 with serine (P3145); a substitution of threonine at position 305 with serine (T305S); a double substitution of threonine at position 305 with serine and isoleucine at position 315 with valine (T305S+I315V); a substitution of histidine at 313 with tyrosine (H313Y); a substitution of alanine at 302 with serine (A302S), or one or more of the foregoing in combination.

In one embodiment, the mutation is selected from one of the following single amino acid substitutions: Q310H, I315V, P3145, T305S, H313Y, A302S. In one embodiment, the mutation is Q310H.

In one embodiment, the method further comprises detecting a substitution of aspartate with glutamate at position 239 (D239E) of the HA protein (an HA D239E mutation). In another embodiment, the method further comprises detecting a substitution of the histidine at position 274 of the neuraminidase protein (NA) with a tyrosine (an NA H274Y mutation).

In a further embodiment of the method for identifying a virulent strain of a virus, the method comprises determining the case fatality rate for hospitalization (CFR/H), wherein the term CFR/H is the number of people admitted to hospitals for treatment of the virus who subsequently die divided by the total number of people admitted for treatment of the virus, wherein an increase in CFR/H over a period of time indicates a virulent strain of the virus.

In a particularly preferred embodiment, the method is a computer-implemented method for identifying a virulent strain of a virus, the method comprising (i) receiving a first data corresponding to a number of individuals admitted to a hospital for treatment of the virus; (ii) receiving a second data corresponding to a number of individuals who subsequently die after being admitted to the hospital for treatment of the virus; and (iii) determining the case fatality rate for hospitalization (CFR/H) using the first and second data, wherein the CFR/H is determined by dividing the number in the second data by the number in the first data, and wherein an increase in CFR/H over a period of time indicates a virulent strain of the virus.

In one embodiment of the computer-implemented method, the first and second data are received from at least one database.

In one embodiment of the computer-implemented method, the receiving further comprises using a processor, querying the first and second data from the at least one database.

Preferably, the CFR/H ratio is determined using first and second data received from multiple hospitals located in more than one geographic region. In certain embodiments, the geographic region is a county, a state, a province, a country, or a continent. In a preferred embodiment, the geographic region is a state or a country.

In certain embodiments, the period of time is measured in weeks or months. In one embodiment, the period of time is from 4 to 8 weeks, 4 to 12 weeks, or 4 to 24 weeks.

In one embodiment, the method further comprises identifying a change in one or more of amino acids 301 to 316 of the HA protein of the virus isolated from fatal cases relative to either (i) virus isolated from nonfatal cases earlier in time or (ii) virus isolated from contemporaneous nonfatal cases, wherein a change in one or more of amino acids 301 to 316 of the HA protein of the virus isolated from fatal cases indicates a more virulent strain of the virus.

In one embodiment, the method further comprises identifying a change in one or more of amino acids 301 to 316 of the HA protein of the virus relative to the reference sequence GAINTSLPFQNIHPIT (SEQ ID NO:5).

The invention also provides a method for diagnosing a virulent viral infection in a human subject, the method comprising detecting a virulent strain of a virus according to the methods described herein.

The invention also provides a kit or pack comprising a set of primers for detecting a mutation in the RNA sequence of the virus encoding amino acid residues 301 to 316 of the hemagglutinin protein (HA). In another embodiment, the kit comprises an antibody for detecting a mutation in amino acid residues 301 to 316 of the hemagglutinin protein (HA) of the virus. In one embodiment, the kit contains both a set of primers and an antibody. In a further embodiment, the kit contains a reference sequence which may be in the form of an RNA molecule, a DNA molecule, a protein, or a peptide. Alternatively, the sequence is included in written form, along with a set of instructions for using the kit in accordance with the methods of the invention.

The invention also provides a system for performing the computer-implemented method for identifying a virulent strain of a virus comprising a memory and a processor coupled to a memory and configured to receive a first data corresponding to a number of individuals admitted to a hospital for treatment of the virus and a second data corresponding to a number of individuals who subsequently die after being admitted to the hospital for treatment of the virus. In one embodiment, the processor is configured to query the first and second data from at least one database.

The invention also provides a computer program product comprising a machine-readable medium storing instructions that, when executed by at least one processor, cause the at least one processor to perform operations comprising (i) receiving a first data corresponding to a number of individuals admitted to a hospital for treatment of the virus; (ii) receiving a second data corresponding to a number of individuals who subsequently die after being admitted to the hospital for treatment of the virus; and (iii) determining the case fatality rate for hospitalization (CFR/H) using the first and second data by dividing the number in the second data by the number in the first data. In one embodiment, the receiving operations further comprise using a processor, querying the first and second data from the at least one database.

### BRIEF DESCRIPTION OF THE FIGURES

FIG. 1: Patterns of changes in clinical outcomes of the pandemic (H1N1) 2009 indicate increasing disease severity. p values were calculated using 2-tailed Fisher's exact test. (A) Weekly case fatality ratios of cumulative counts deaths to hospitalizations (CF/H) for patients diagnosed with H1N1 infection in United States, (USA) California (CAL), England (UK), Australia (AUS), and Canada (CAN) during the July to August 2009 time period. The data show an increase in fatality rates during the most recent reporting periods for the U.S. overall and for the U.K., Australia, and Canada. (B). Different patterns of CF/H in California and Florida. (C) Increasing mortality rates at the peaks of outbreaks during three consecutive waves of the pandemic (H1N1) 2009 in New York City (April 23-June 23), California (July 3-July 30), and Florida (July 30-September 29). (D-G) Monthly CF/H for patients diagnosed with H1N1 infection in Australia (D, E), England (F), and Canada (G) during the July-September time periods. The data indicate increasing fatality rates during the reporting period. In Australia, data point stratification was performed by time periods (D) or by the first time point reporting 1000 new consecutive hospitalization cases (E). (H-K) Monthly CF/H for patients diagnosed with H1N1 infection in United States. In United States, the CDC implemented new reporting requirements beginning Aug. 30, 2009. Data analysis was performed separately based on old CDC reporting protocol (shown in H, from July 30^{th} to September 3^{rd}) and four weeks based on the new protocol (shown in I-K, August 30^{th} to September 26^{th}). (L-M) Weekly CF/H for patients diagnosed with H1N1 infection in Japan. (N) A statistically higher proportion of patients required hospitalization in Japan. (O) A statistically higher proportion of hospitalized patients required treatment in the intensive care units during the July to August time period in the U.K. (England) and Canada.

FIG. 2: Patterns of changes in clinical outcomes of the pandemic (H1N1) 2009 indicate increasing disease severity. A-D. Analysis of clinical features of the pandemic (H1N1) 2009 during the winter season in Australia reveals stable levels of hospitalization cases (B) and hospitalization rates (C) and significantly rising death rates (A, D).

FIG. 3: Patterns of emergence of the hemagglutinin (HA) mutations during the pandemic (H1N1) 2009. (A) Instances of mutations within the 301-316 amino acid segments of HA reported in the NCBI database (Aug. 31, 2009 freeze) during the indicated time periods in Mexico, California, New York, and Canada. Note a concomitant marked increase of mutations during the July to August time periods. (B) Instances of representation of the wild-type sequence (Mexico) and the sequences containing mutations in the 301-316 amino acid segments of HA reported in the NCBI database (Aug. 31, 2009 freeze). Note that mutations constitute 8% of all reported cases and the Q310H mutation is reported in 4% of total population and 51% of mutated virus. (C) Timeline of emergence of two major variants of the HA 301-316 sequence indicates that a “silent wave” of the (H1N1) pandemic was occurring during the 2008 flu season. (D) Global distribution analysis of all mutations of the 301-316 amino acid segments of HA gene reported in the NCBI database (Aug. 31, 2009 freeze). Months indicate time of the first mutation report in corresponding countries.

FIG. 4: Patterns of global and regional associations between the emergence of the HA 301-316 segment mutations and increasing death rates during the pandemic (H1N1) 2009. (A) Timeline of reporting in the NCBI database of HA sequences flagged by the three variants: (1) a serine at position 220 (S220), (2) a threonine at position 220 (T220), and/or (3) a substitution of the glutamine at position 310 with histidine (Q310H). All corresponding entries of human cases isolated during the pandemic (H1N1) 2009 are sorted by the GenBank accession numbers. As of Sep. 16, 2009, T220 and S220 variants constitute 41.5% and 58.5% of the sequenced H1N1 virus population; virus isolates containing the Q310H mutation comprise 5.1% of the sequenced population and contain only the HA variant with a serine at position 220 (S220). (B-C) Timeline analysis of reporting in the NCBI database of HA sequences flagged by the three variants described in (A) above: S220, T220, and Q310H. The data show a statistically significant expansion of the Q310H mutation within both the entire sequenced H1N1 virus population (B) and within the HA S220 H1N1 sub-population (C). Percentages of corresponding variants per 200 new consecutive reported instances of HA sequences are shown. (D) An increasing rate of the HA 301-316 segment mutations reported in the NCBI database is associated with an increase in reported deaths associated with pandemic (H1N1) 2009. (E) Cumulative worldwide death rates of the pandemic (H1N1) 2009 are significantly lower compared to estimated death rate of patients with the Q310H HA mutation. (F) Timeline of increasing death rates of reported worldwide cases of the pandemic (H1N1) 2009. (G) Timeline association in New York between reported instances of H1N1-related deaths and HA 301-316 sequence mutations. Note that reported dates of collection of samples with subsequently detected mutations precede dates of spikes in H1N1-associated deaths.

FIG. 5: Analysis of the distribution of CDC reported H1N1-associated deaths in HHS-defined regions of the United States segregated on the basis of detection of the new 2009 HA 301-316 segment sequence mutations (A) or the HA triple mutation (B). The vast majority of the HA triple mutation cases were collected in 2008 with a small number of cases reported in 2009. Note that in comparison to regions with no cases of HA mutations or regions with HA triple mutation, regions with reported instances of the new 2009 HA 301-316 sequence mutations account for disproportionally large fractions of H1N1-realted deaths and have significantly higher H1N1-associated mortality rate (p=1.00E-09; 2-tailed Fisher's exact test). Panel (C) shows pediatric deaths.

FIG. 6: Patterns of global and regional associations of timelines of emergence of the HA 301-316 mutations and increasing death rates during the pandemic (H1N1) 2009. Timeline association in Canada (A), Mannitoba (B), and Brazil (C, D) between reported instances of H1N1-related deaths and HA 301-316 sequence mutations. Note that in all cases the collection of samples with subsequently detected mutations precedes the increase in H1N1-associated deaths.

### DETAILED DESCRIPTION OF THE INVENTION

The present invention is directed to methods for identifying a virulent strain of a virus and related diagnostic and prognostic methods. The virus is preferably an RNA virus, preferably of the family Orthomyxoviridae. In a one embodiment, the virus is an influenza virus. In certain embodiments the influenza virus is an influenza A, B, or C virus. In a preferred embodiment the virus is an influenza A virus. In certain embodiments, the influenza A virus is characterized as a particular strain, subtype or serotype (these terms are used interchangeably herein) based on the antibody response to the two viral proteins, hemagglutinin (HA) and neuraminidase (NA). In particular embodiments, the serotype is selected from H1N1, H2N2, H3N2, H5N1, H7N7, H1N2, H9N2, H7N2, H7N3, or H10N7. In a one embodiment, the serotype is H1N1, H2N2, H3N2, H5N1, H7N7, or H1N2. In another embodiment, the serotype is H1N1, H2N2, H3N2, or H5N1. In a preferred embodiment, the serotype is H1N1.

The term virulent is used in accordance with its normal and customary meaning in reference to the pathogenicity of a virus in a population. One commonly used measure of virulence is the case fatality rate. The case fatality rate (“CFR”) is the proportion of a population of infected individuals who die. Death may result either directly from the primary viral infection or indirectly from a secondary bacterial infection, or from complications arising from either the primary or the secondary infection. Thus, the CFR is represented numerically as the number of persons dying from a disease (i.e., viral infection) divided by the number of persons diagnosed with the disease. In the context of the present invention, a higher CFR indicates a more virulent strain of the virus. The CFR is commonly represented as a percentage.

In one embodiment, the methods of the invention comprise detecting a mutation within a particular segment of the hemagglutinin protein (HA) of the virus, namely within amino acid residues 301 to 316, wherein the detection of one or more mutations within this region indicates a virulent strain of the virus. The HA protein is a glycoprotein found on the surface of the virus. As used herein, the numbering of the amino acid residues is with reference to SEQ ID NO:25. Using this sequence as a reference, the corresponding amino acids in any viral HA protein can be easily identified using tools common in the art. For example, by performing a pair-wise sequence alignment between the reference sequence and the HA amino acid sequence of interest.

In accordance with the methods of the invention, a mutation within amino acid residues 301 to 316 of an HA protein from a virus isolated from the cells or tissues of an infected subject (“the subject HA protein”) is detected with respect to a reference sequence. In one embodiment, the reference sequence comprises amino acids 301 to 316 of an HA protein isolated from an outbreak of the virus earlier in time than the virus from which the subject HA protein was isolated. In accordance with particular aspects of this embodiment, the outbreak of the virus earlier in time occurred 0-3 months, 3-6 months, 6-9 months, 9-12 months, 12-15 months, 15-18 months, or 18-21 months earlier. In one embodiment, the outbreak earlier in time occurred within the same pandemic as the virus from which the subject HA protein was isolated.

In one embodiment, the reference sequence comprises amino acids 301 to 316 of an HA protein isolated from a viral outbreak in a different geographical region from the subject HA protein. In one embodiment, the reference sequence comprises amino acids 301 to 316 of an HA protein isolated from a viral outbreak in a different country than the subject HA protein.

In one embodiment, the reference sequence comprises amino acids 301 to 316 of an HA protein isolated from an earlier influenza pandemic. In a particular embodiment, the reference sequence comprises amino acids 301 to 316 of an HA protein first isolated from Mexico in 2009 during the early period of the swine flu pandemic of 2009 to 2010. In accordance with this embodiment, the reference sequence comprises SEQ ID NO:5.

Preferably, the one or more mutations are detected in a virus isolated directly from the cells or tissues of an infected subject, without having been propagated in vitro. In another embodiment, the one or more mutations are detected in a virus that has been propagated in vitro.

The subject in accordance with the methods of the invention is preferably a human subject, but the subject may also be a pig, a bird, a cow, a dog, a cat, a ferret, or a non-human primate.

In particular embodiments the HA protein is further characterized by its subtype or serotype (these terms are used interchangeably herein) as any one of H1, H2, H3, H4, H5, H6, H7, H8, H9, H10, H11, H12, H13, H14, H15, or H16. In a preferred embodiment, the HA protein is an H1 or an H5 HA protein.

Preferably, the present methods utilize the large volume of data that is generated and made available by governmental, intergovernmental, and nongovernmental agencies during a viral outbreak or pandemic. This data includes sequence data obtained from viral isolates of infected persons or animals. The data also includes information regarding the number of persons hospitalized with a viral infection, their diagnosis, and outcome, including death.

The detection of a mutation in the viral HA protein in accordance with the methods of the invention can be accomplished using techniques routine in the art. In one embodiment, a mutation is detected by comparing the subject sequence to a reference sequence using art-recognized methods. In one embodiment, a mutation is detected using techniques based on nucleic acid hybridization or techniques based on specific amplification using the polymerase chain reaction (PCR based techniques). In another embodiment, the one or more mutations is detected by comparing the subject sequence to a reference sequence using in silico methods. Such methods include sequence search, comparison, and alignment tools known in the art. Non-limiting examples of such tools include BLAST (Basic Local Alignment Search Tool, Altschul S. F., et al. 1990); CS-BLAST (Biegert A. and Soding J. 2009); FASTA; GGSEARCH/GLSEARCH Global:Global (GG), Global:Local (GL); HMMER (Durbin R. et al., (1998); HHpred/HHsearch (Soding J. 2005); PSI-BLAST (Altschul S. F. et al., 1997); SAM (Karplus K. and Krogh A. 1999); and SSEARCH (a Smith-Waterman search). Further non-limiting examples include AlignMe (Khafizov et al. 2010); Bioconductor Biostrings::pairwiseAlignment (Aboyoun 2008); BioPerl dpAlign (Chan 2003); BLASTZ,LASTZ (Schwartz et al. 2004, 2009); DNADot (Bowen 1998); DOTLET (Pagni and Junier 1998); FEAST (Hudek and Brown 2010); GGSEARCH, GLSEARCH Global:Global (GG), Global:Local (GL) (Pearson 2007); JAligner (Moustafa 2005); LALIGN (Pearson 1991); mAlign (Powell, Allison Dix 2004); SABERTOOTH (Teichert et al. 2009); SEQALN (Waterman and Hardy 1996); and YASS (Noe and Kucherov 2003-2007). This list is not exhaustive and those of skill in the art will be aware of additional tools, for example, those designed for multiple sequence alignments and motif finding, that could also be adapted for use in the methods described by the invention.

In a preferred embodiment of the methods described herein, the one or more mutations in the HA protein is detected by identifying the mutations in the sequences of HA proteins isolated from the cells or tissues of infected subjects, preferably human subjects, and deposited into one or more searchable public databases, i.e., during a current outbreak or pandemic. In accordance with this embodiment, at least part of the method is carried out on a machine, i.e., a computer, which executes instructions to carry out a comparison of one or more viral HA sequences deposited in the public database with a reference sequence to identify one or more mutations in each of the database sequences. The amino acid and nucleic acid sequences of known HA proteins are commonly available in various public databases maintained by governmental and nongovernmental organizations such as the National Center for Biotechnology Information (NCBI) and the Swiss Institute of Bioinformatics.

The one or more mutations detected in the HA sequence according to the invention is preferably an amino acid substitution. In one embodiment, the reference sequence is SEQ ID NO:5 and the mutation detected in the HA protein of the virus isolated from the cells or tissues of the subject is selected from a substitution of glutamine at amino acid position 310 with histidine (Q310H); the triple substitution of threonine at position 305 with serine, isoleucine at position 312 with valine, and isoleucine at position 315 with valine (T305S+I312V+I315V); a substitution of isoleucine at position 315 with valine (I315V); a substitution of proline at 314 with serine (P3145); a substitution of threonine at position 305 with serine (T305S); a double substitution of threonine at position 305 with serine and isoleucine at position 315 with valine (T305S+I315V); a substitution of histidine at 313 with tyrosine (H313Y); a substitution of alanine at 302 with serine (A302S), or one or more of the foregoing in combination. In accordance with this embodiment, the first amino acid of SEQ ID NO:5 is designated number 301 and the other amino acids are sequentially designated 302-316. The specific mutations referred to here are further described in Table 1, infra.

In one embodiment, the method further comprises detecting one or more additional amino acid substitutions outside of amino acid residues 301-316 of the HA protein. In a specific embodiment, the method further comprises detecting a substitution of aspartate with glutamate at position 239 of the HA protein.

In one embodiment, the method further comprises detecting one or more additional amino acid substitutions in the neuraminidase (NA) protein. Preferably, the one or more additional amino acid substitutions in the NA protein confer resistance to one or more antiviral drugs. In a specific embodiment, the mutation is a substitution of the histidine at position 274 with a tyrosine (H274Y).

In one embodiment, the method comprises determining the number of persons hospitalized for infection by the virus who die from the infection compared to the total number of persons hospitalized for infection. This is referred to as the case fatality rate for hospitalization, or “CFR/H”. An increasing CFR/H over a period of time indicates a virulent strain of the virus.

Preferably, the CFR/H is determined for two or more geographic regions. In specific embodiments the CFR/H is determined in two or more counties, states, provinces, countries, or continents. In a preferred embodiment, the CFR/H is determined in two or more countries, preferably at least 3 countries. In specific embodiments, the CFR/H is determined in 2, 3, 4, 5, 6, 7, 8, 8, or 10 different countries. Preferably, at least two of the countries are located on different continents.

In certain embodiments, the period of time is measured in weeks. In other embodiments, the period of time is measured in months or years. In specific embodiments the period of time is 2, 3, 4, 5, 6, 7, 8, 9, 10, 11, 12, 14, 16, 18, or 20 weeks; or from 2-4, 4-6, 6-8, or 8-12 weeks. In other embodiments the period of time is 2, 3, 4, 5, 6, 7, 8, 9, 10, 11, 12, 13, 14, 15, 16, 17, 18, 19, 20, 21, 22, 23, or 24 months. In one embodiment the period of time is 2, 4, 6, 8, 10, 12, 14, 16, or 18 months.

The present invention also provides methods for diagnosing a virulent viral infection in a human subject, the method comprising detecting a virulent strain of a virus according to the methods described herein. The invention also provides methods of treating of human subject diagnosed with a virulent strain of virus, the methods comprising administering one or more antiviral drugs to the subject. In one embodiment, the one or more antiviral drugs is selected from a neuaminidase inhibitor, including for example, oseltamivir (TAMIFLU) and zanamivir (RELENZA). In another embodiment, the one or more antiviral drugs is selected from an M2 inhibitor, including for example, adamantanes such as amantadine and rimantadine. In a further embodiment, the one or more antiviral drugs is selected from oseltamivir, zanamivir, amantadine, and rimantadine.

The invention also provides a kit or pack containing reagents and instructions for carrying out the claimed methods. In one embodiment, the kit contains a set of primers for detecting a mutation in the RNA sequence of the virus encoding amino acid residues 301 to 316 of the hemagglutinin protein (HA). In another embodiment, the kit contains an antibody for detecting a mutation in amino acid residues 301 to 316 of the hemagglutinin protein (HA) of the virus. In another embodiment, the kit contains both a set of primers and an antibody. In a further embodiment, the kit contains a reference sequence which may be in the form of an RNA molecule, a DNA molecule, a protein, or a peptide. Alternatively, the sequence is included in written form, along with a set of instructions for using the kit in accordance with the methods of the invention.

In a preferred embodiment of a method of the invention, the method is a computer-implemented method for identifying a virulent strain of a virus, the method comprising (i) receiving a first data corresponding to a number of individuals admitted to a hospital for treatment of the virus; (ii) receiving a second data corresponding to a number of individuals who subsequently die after being admitted to the hospital for treatment of the virus; and (iii) determining the case fatality rate for hospitalization (CFR/H) using the first and second data, wherein the CFR/H is determined by dividing the number in the second data by the number in the first data, and wherein an increase in CFR/H over a period of time indicates a virulent strain of the virus. In one embodiment of the computer-implemented method, the first and second data are received from at least one database. In one embodiment of the computer-implemented method, the receiving further comprises using a processor, querying the first and second data from the at least one database.

The invention also provides a system for performing the computer-implemented method for identifying a virulent strain of a virus comprising: a memory; a processor coupled to a memory and configured to receive a first data corresponding to a number of individuals admitted to a hospital for treatment of the virus; receive a second data corresponding to a number of individuals who subsequently die after being admitted to the hospital for treatment of the virus. In one embodiment, the processor is configured to query the first and second data from at least one database.

The invention also provides a computer program product comprising a machine-readable medium storing instructions that, when executed by at least one processor, cause the at least one processor to perform operations comprising (i) receiving a first data corresponding to a number of individuals admitted to a hospital for treatment of the virus; (ii) receiving a second data corresponding to a number of individuals who subsequently die after being admitted to the hospital for treatment of the virus; and (iii) determining the case fatality rate for hospitalization (CFR/H) using the first and second data according to the methods of the invention. In one embodiment, the receiving operations further comprise using a processor, querying the first and second data from the at least one database.

The following diagram illustrates an exemplary method for identifying a virulent strain of a virus, according to some embodiments of the present invention. The method can be performed by a system having a processor and a memory. As shown in the diagram, a data corresponding to a number of individuals admitted to a hospital for treatment of the virus can be received. A data corresponding to a number of individuals who subsequently die after being admitted to the hospital or treatment of the virus can also be received. The processor can be also configured to query such data. Both sets of data can be further configured to be stored in a database. The processor can be configured to communicate with the database either via a wired, wireless, wireline, or any other type of communication method. Using the first and second data, a case fatality rate for hospitalization (CFR/H) can be determined. Such rate can be determined by dividing the data corresponding to the number of people admitted to hospitals for treatment of the virus who subsequently die divided by the total number of people admitted for treatment of the virus, which is part of the second data received by the processor. Using the CFR/H, the virulent strain of the virus can be identified. In some embodiments, an increase in the CFR/H over a period of time can indicate a virulent strain of the virus.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they may include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The subject matter described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

### EXAMPLES

Early epidemiological observations and experimental studies suggested that the novel influenza A (H1N1) virus had a significant pandemic potential. This was based on the apparent high transmissibility of the virus among humans and the genetic diversity of the virus. In the present study, we evaluated the practical utility of real-time analysis of the large volume of data generated by the global monitoring of the H1N1 pandemic. The data include clinical, epidemiological, and genomic data. The goal is to rapidly characterize the clinical and genomic evolution of the pandemic in order to provide physicians and public health officials with the data and tools necessary to confine the spread of a virulent strain of the virus, either as it evolves during a pandemic or during the initial outbreak period, before the pandemic potential has been realized.

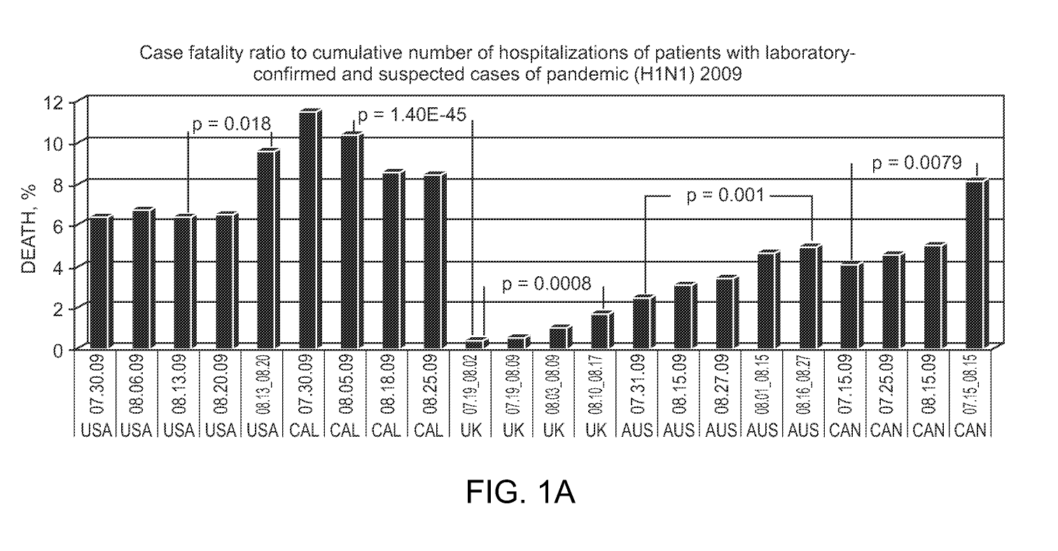

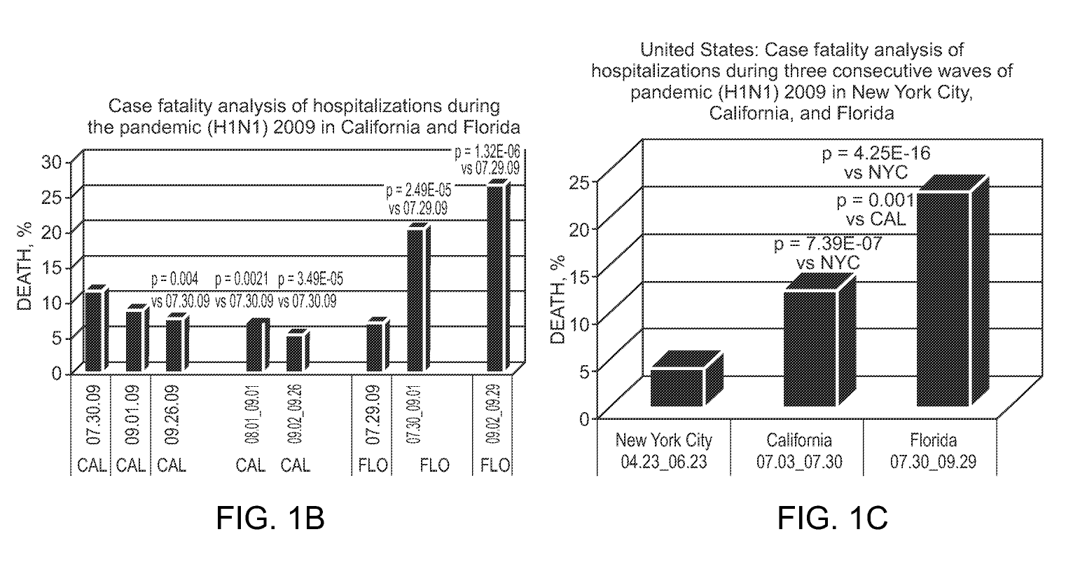

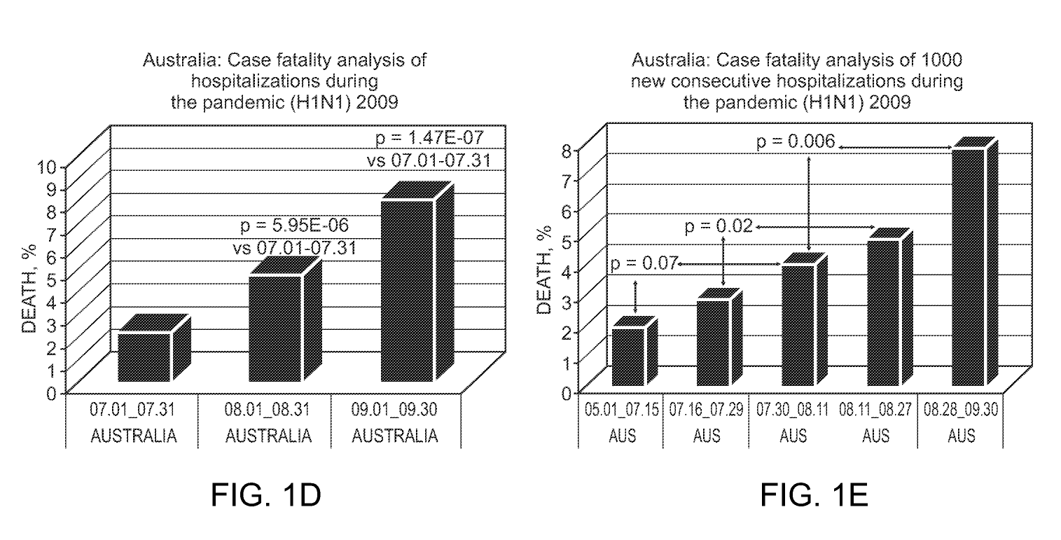

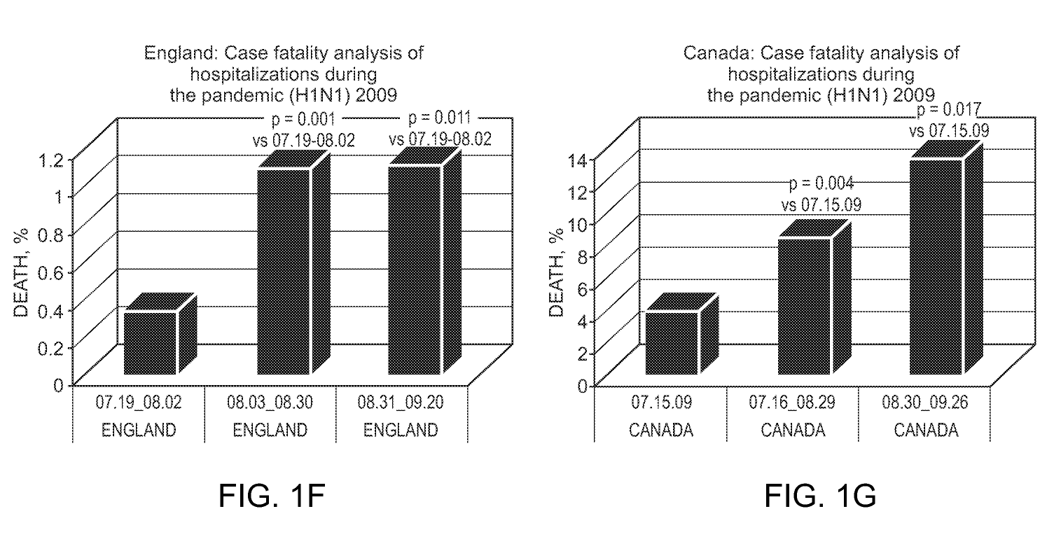

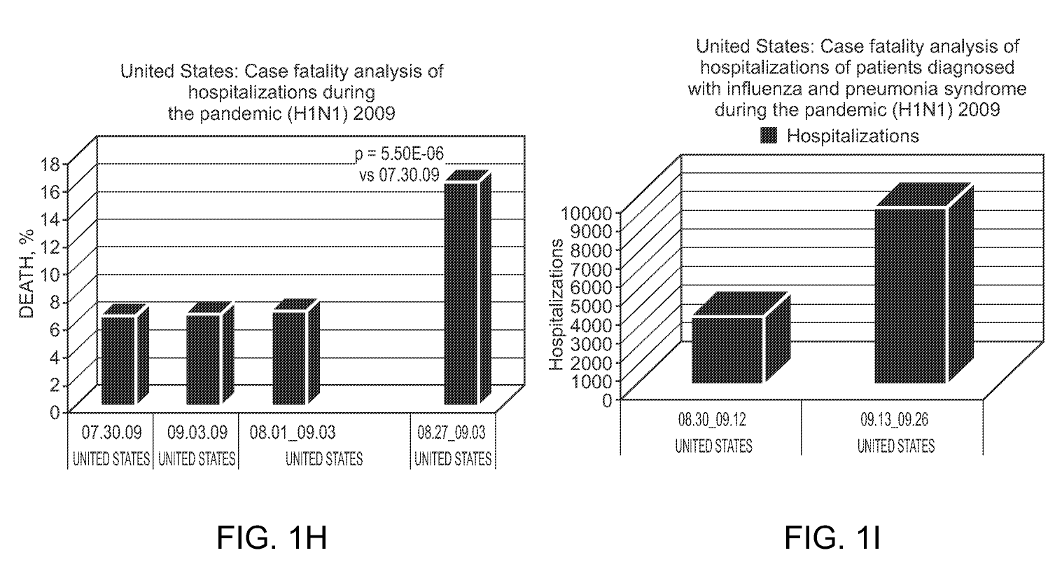

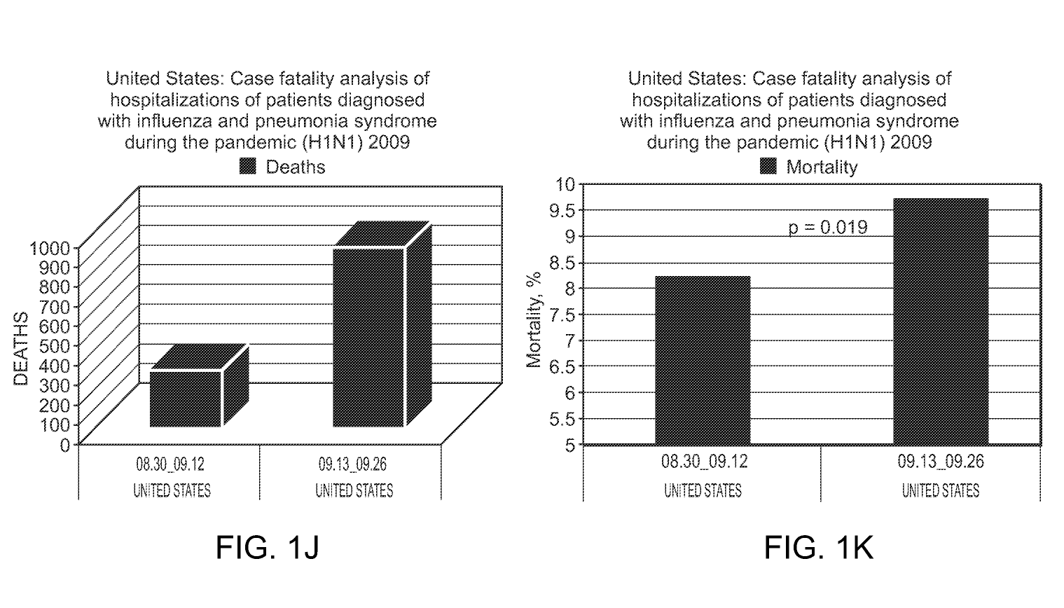

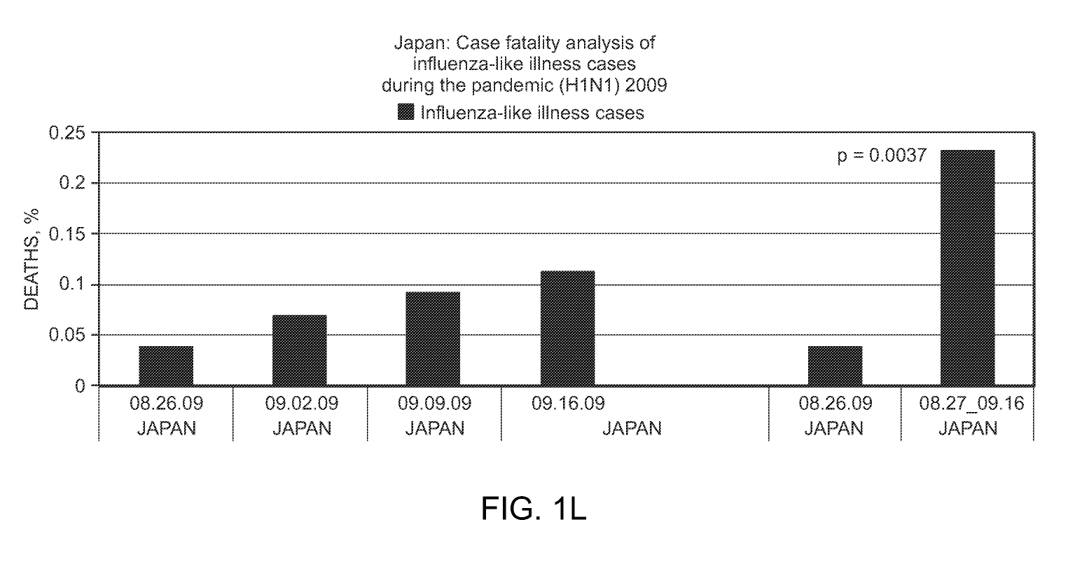

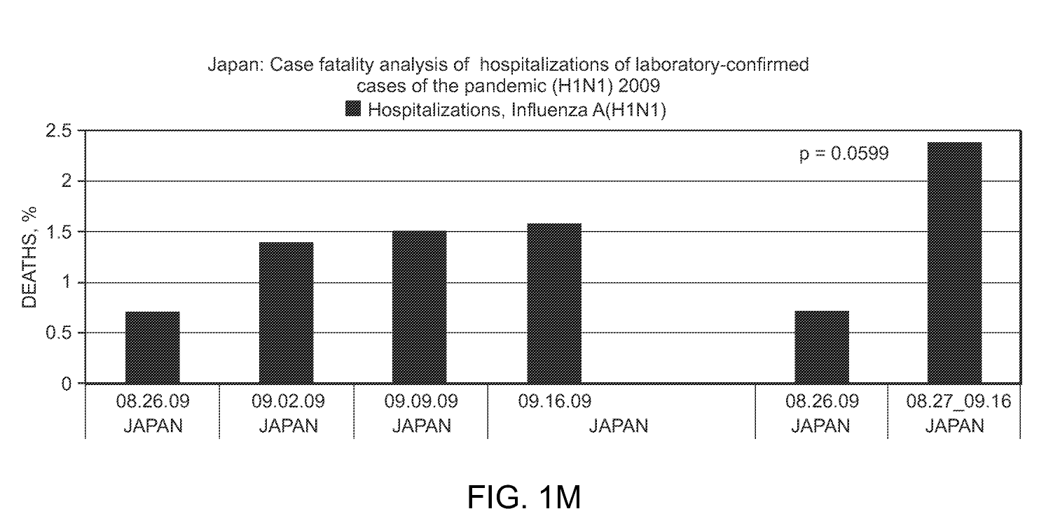

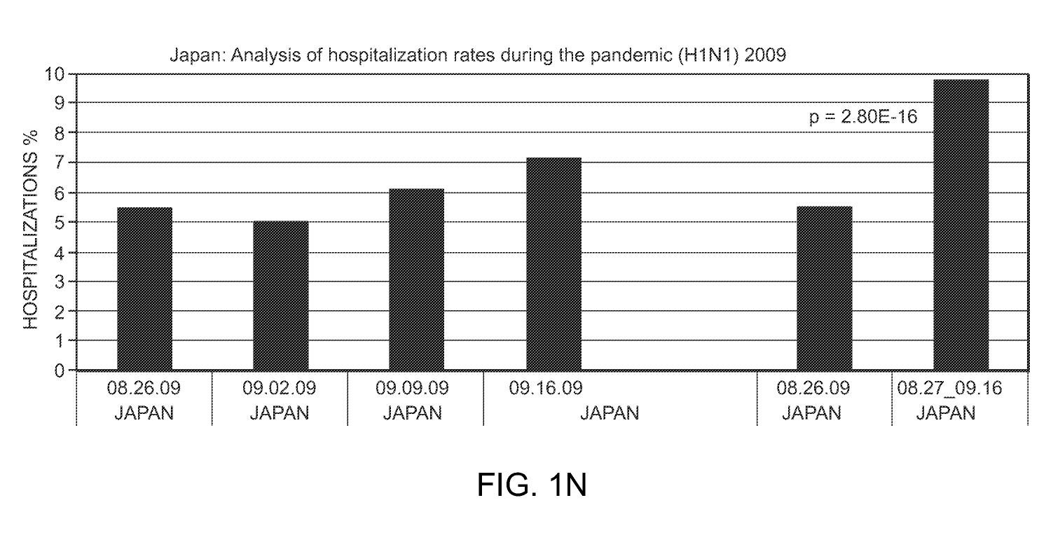

First, we assessed the clinical severity of the pandemic (H1N1) 2009 by analyzing the case fatality ratio compared to the number of hospitalizations (“CF/H”) in four developed countries with robust health care systems, the United States (U.S.), Canada, the United Kingdom (U.K.), and Australia. The raw data for this analysis was provided by the official reports of the public health agencies of these. Similar outcomes for the CF/H were expected among these four countries because each was assumed to have a similarly sophisticated health care system. Surprisingly, the analysis revealed marked variability in the clinical outcomes, both between countries and within the U.S. (FIG. 1A-1C). For example, in the United Kingdom, the CF/H index was only 0.34% during the study period from July to August 2009. In contrast, this index value was 2.9% in Australia, 4.6% in Canada, and 6.7% in the U.S. In certain states within the U.S., the index was even higher, as exemplified by the 10.3% value for California and the even higher rates for Florida (FIG. 1B-1C).

Closer analysis indicated an increase in the CF/H index over time, with fatality rates increasing during the most recent reporting periods. See FIG. 1C-10. This trend was observed in the United States (“U.S.”), Australia, the United Kingdom (“U.K.”), Canada, and Japan. Although the variability in the index value between different countries can be explained in part by differences in the efficiency of the corresponding health care systems, the consistent statistically significant time dependency of the increase in the CF/H index within the same country suggests an increase in the clinical severity of later cases compared to earlier cases. Thus, an increasing CF/H index is associated with increasing disease severity.

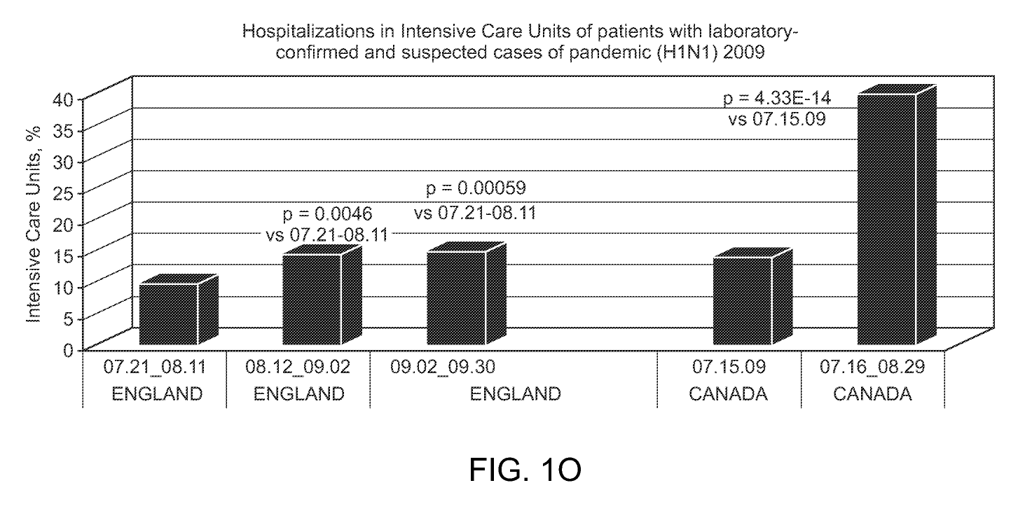

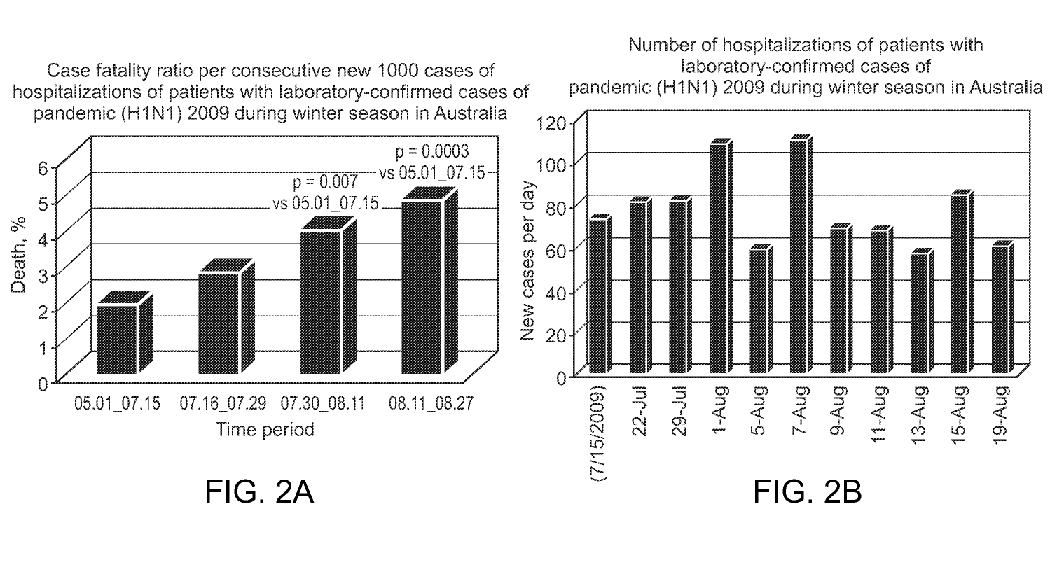

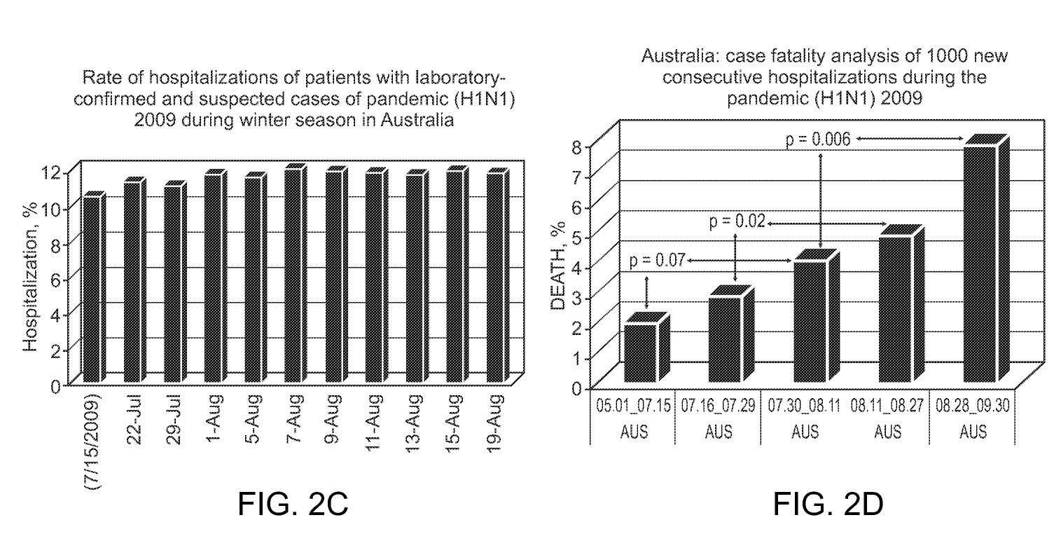

The association of the CF/H index with disease severity is further supported by a detailed timeline analysis of the hospitalizations and death rates in Australia during the winter season of the H1N1 2009 pandemic (FIG. 2). While both the number of hospitalizations and hospitalization rates remained stable, the CF/H index progressively increased, indicating that patients being admitted in the latter months of the pandemic presented with a more severe form of the disease.

One mechanism underlying an observed increased in disease severity during a pandemic is acquired mutations by the virus, e.g, mutations that increase transmissibility and/or virulence. In general, the identification of particular mutations having clinical significance is hampered by the high rate of mutation observed in the influenza viruses. This high rate of mutation means that it is difficult to predict where in the viral genome the clinically important mutations will occur. An analysis of the entire genome for relevant mutations during the course of a pandemic would be too time-consuming and costly to be of practical use.

Here we demonstrate that analysis of a short stretch of amino acids within the hemagglutinin (HA) protein detected mutations that correlated with increased clinical severity as measured by the CF/H index. This segment has only two amino acid substitutions compared to the 1918 H1N1 virus. These are a substitution of threonine with serine at position 305 (T305S) and a substitution of isoleucine with valine at position 315 (I315V). Table 1 shows the results of a systematic survey and manual curation of the National Center for Biotechnology Information (“NCBI”) H1N1 sequence database (Aug. 31, 2009 freeze).

TABLE 1
H1N1 hemagglutinin (HA) sequences of amino acids
301-316 in the NCBI database (Aug. 11, 2009 freeze)

| | SEQ | No. of cases |
|---|---|---|
| | ID | (total)/No. |
| Hemagglutinin 301-316 sequence | NO: | of countries |
| G A I N T S L P F H N I H P I T | 1 | 35/10 |
| G A I N S S L P F Q N V H P V T | 2 | 30/4 |
| (2008 variant) | | |
| G A I N T S L P F Q N I H P V T | 3 | 7(1)/1 |
| (old “reassortment” variant) | | |
| G A I N T S L P F Q N I H S I T | 4 | 5/4 |
| G A I N T S L P F Q N I H P I T | 5 | 839/Global |
| (2009 Mexico) | | |
| G A I N S S L P F Q N I H P V T | 6 | 0/0 |
| (1918 pandemic) | | |
| G A I N T S L P F X N I H P I T | 7 | 6/1 |
| G A I N T S L P F Q N I Y P I T | 8 | 2/2 |
| G S I N T S L P F Q N I H P I T | 9 | 2/1 |
| G X I N T S L P F Q N I H P I T | 10 | 1/1 |

The Top 4 mutations were identified in at least 5 independent reports from at least 3 distinct geographical locations; The list of countries with reported cases of HA 301-316 sequence mutations is as follows:
Q310H: USA (21); Italy (4); China (2); Brazil (2); Finland (1); Japan (1); France (1); Canada (1); Taiwan (1); Singapore (1)
T305S + 1312V + I315V: Spain (9); Japan (5); USA (15); China (1) (Proposed new wild type virus)
I315V: USA (7; 1 in 2009)
P314S: Italy (2); Hong Kong (1); Spain (1); USA (1); Wild type virus (Mexico): Worldwide
T305S + 1315V: 1918 pandemic: No instances reported
Q310X: Canada (6)
H313Y: Philippines (1); USA (1)
A302S: Brazil (2)
A302X: USA (1)

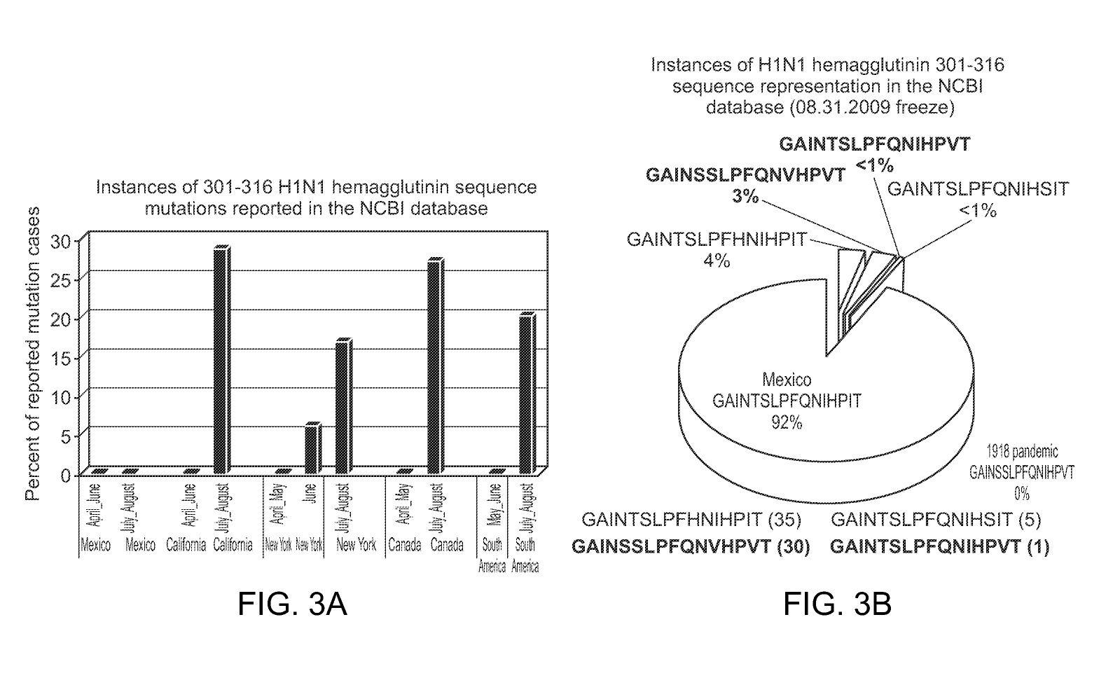

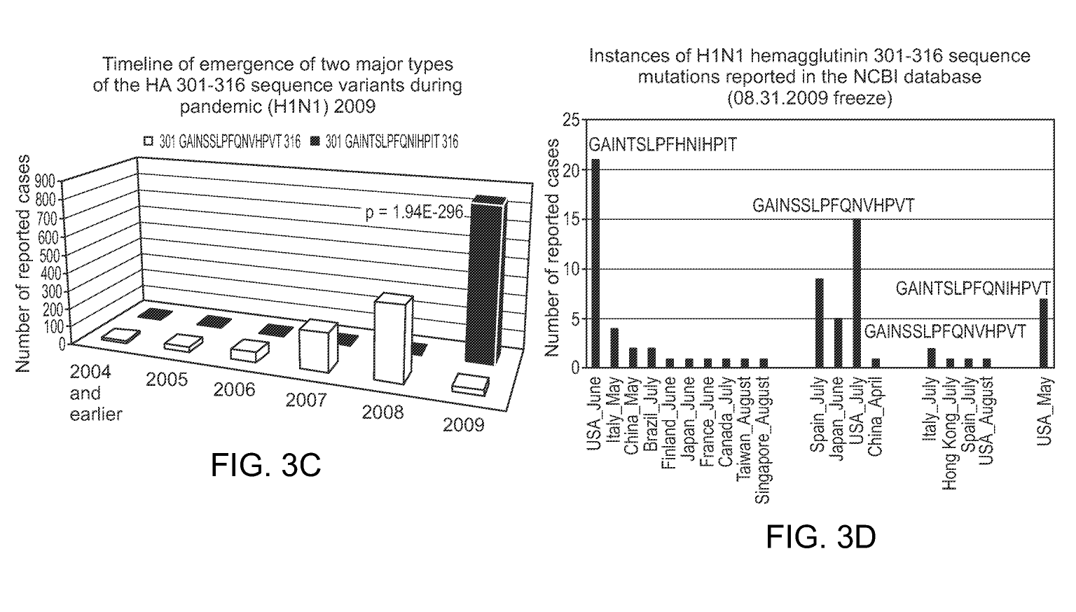

Our analysis identified a global pattern of concomitant increases in the number of HA segment mutations (compared to the sequence of the Mexican isolates, SEQ ID NO: 5) during the July-August reporting period (Table 1 and FIG. 3A). The increase in the number of reported HA segment mutations correlated with the increase in the CF/H index reported in the latter periods of the pandemic in the U.S., Canada, and Australia, as discussed in more detail below.

Consistent with the Mexican origin of the virus, the Mexican variant of the HA 301-316 segment (GAINTSLPFQNIHPIT, SEQ ID NO: 5) represents 92% of the H1N1 sequences deposited in 2009 (FIG. 3B). This segment contained no mutations relative to SEQ ID NO:5 in more than 90% of the H1N1 sequences. About 8% of the sequences did contain mutations in this region. This number probably represents an underestimate since we included in our analysis only incidences of mutations that were identified in at least 5 independent reports from at least 3 distinct geographical locations. Table 1 shows the full catalog of mutations within this segment of the HA protein that were identified during the 2009 pandemic.

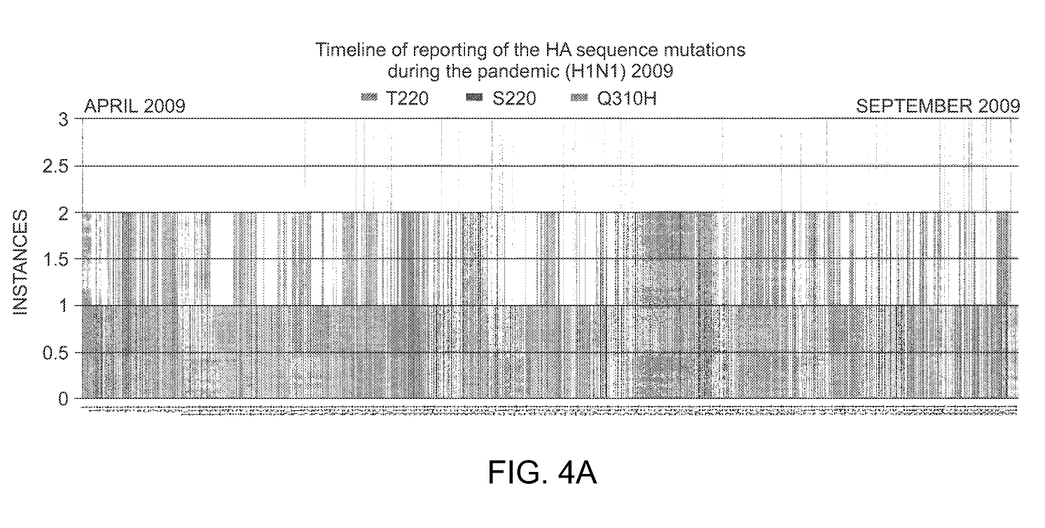

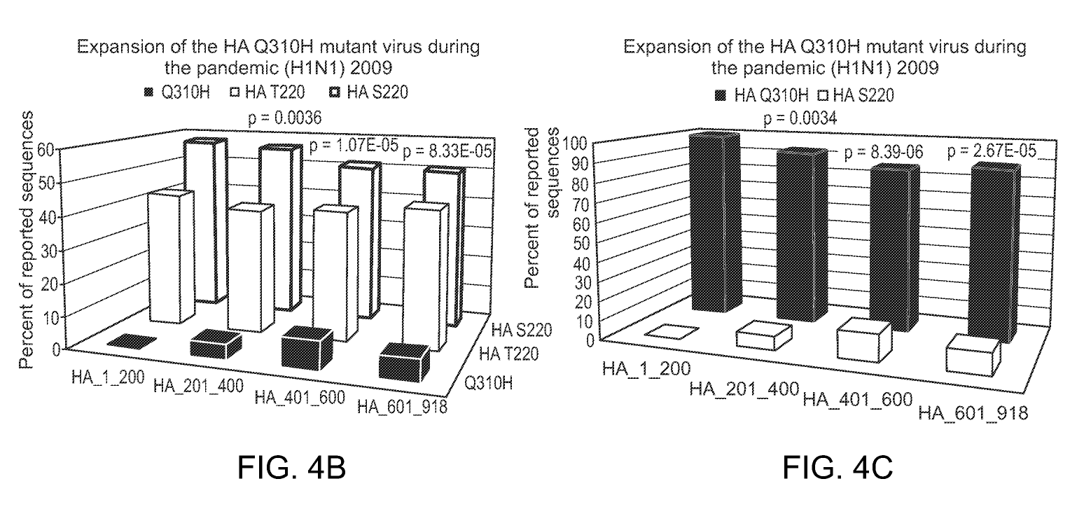

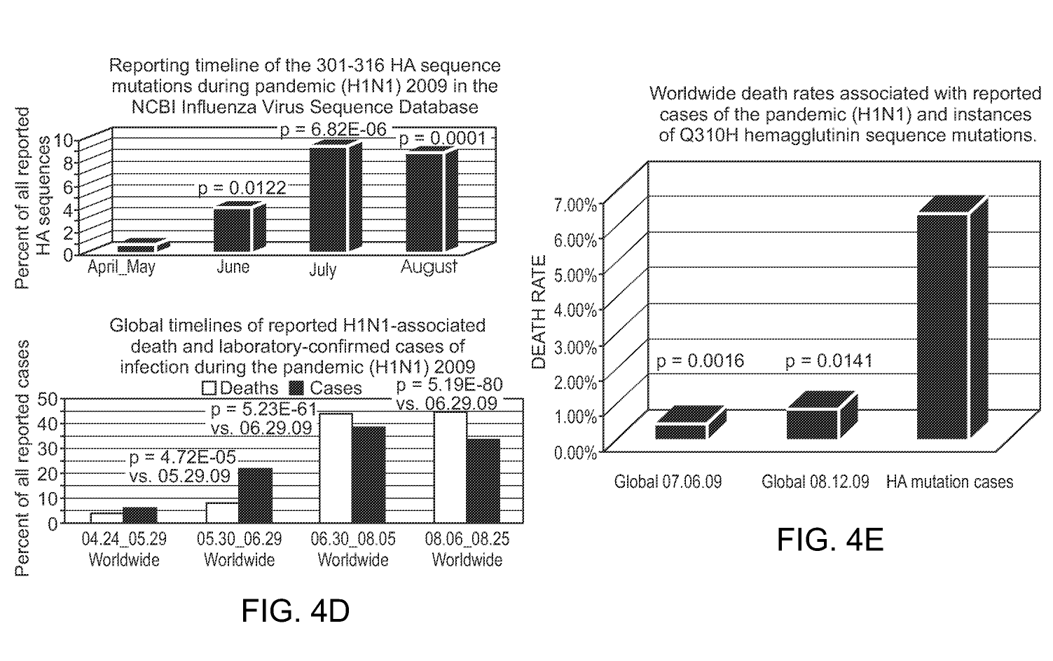

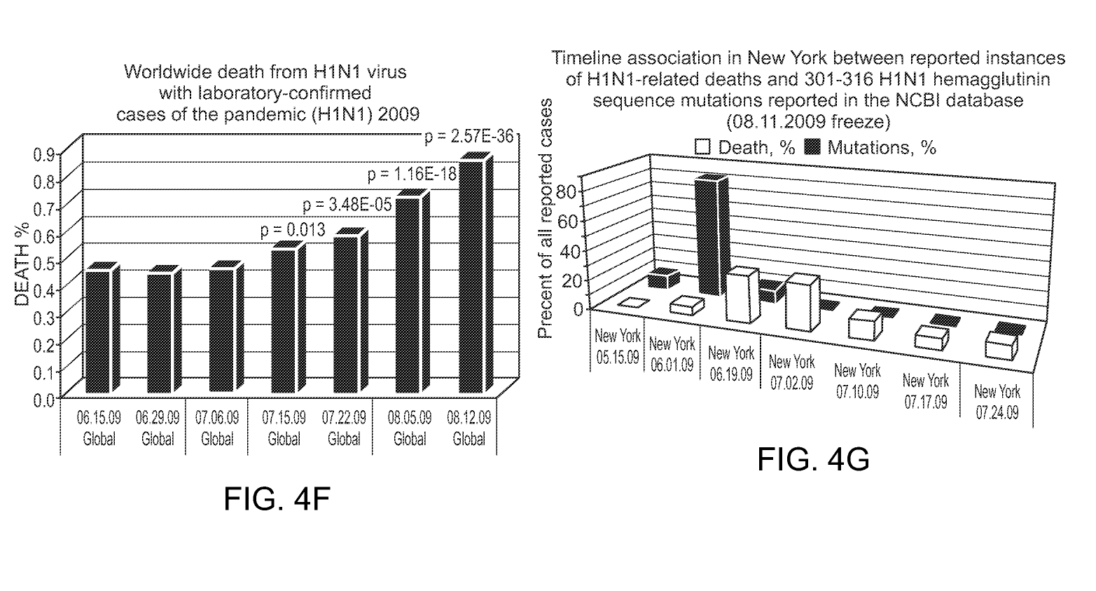

Our data show that the Q310H mutation was first detected in samples collected on Apr. 28, 2009 in the U.S. Within the next few weeks, the Q310H mutation was identified in 7 additional countries. By the end of our analysis period, it was identified in 11 countries and was present in about 4% of all reported cases and about 51% of all newly reported cases (FIGS. 3B, 3D and FIG. 4). A timeline analysis indicated that the Q310H mutantation further expanded within the viral population during the latter months of our analysis period. This data indicates that the Q310H mutation expanded rapidly worldwide.

A second newly identified mutation within the HA 301-316 segment, P314S, is of interest despite the relatively small total number of identified cases (Table 1). Notably, all five viral isolates with the P314S mutation had one additional common HA mutation, D239E. This mutation targets an amino acid residue that has been implicated in both receptor-binding and immunogenicity. In addition, in at least one instance (GQ351314) the HA P314S mutation segregates with the recently identified Oseltamivir-resistance mutation in the NA gene, H274Y. The single segregation event identified here is unlikely to have occurred by chance alone because there are only 5 identified cases of the HA P314S mutation and 5 instances of the NA H274Y mutation in the NCBI database while the cumulative number of reported sequences is in excess of 800 for each. These data indicate that this mutant may be of clinical significance either now or in a future pandemic.

The expansion of the Q310H mutation is in striking contrast with two other prominent HA 301-316 mutations (mutation I315V and the triple mutation T305S+I312V+I315V). All six cases of the I315V mutation submitted to the NCBI database in 2009 were collected in earlier years: 1991 (CY039909), 1976 (CY039991), 2005 (FJ986619), 2006 (FJ986618), and 2007 (FJ986620), (FJ986621). This indicates that these mutations have no direct relationship to the current pandemic. The first samples of 2009 year triple mutant (T305S+I312V+I315V) were collected in January in Spain (9 cases), United States (15), and Japan (5). The last reported incidents of this mutation were collected in March 2009 in China and in June 2009 in the U.S. Multiple instances of this mutation were reported around the world for several years with most cases reported in 2008 (400 cases in 2008 compared to 30 in 2009), suggesting that this marker mutation is not likely to reflect the association with disease severity and may be considered as one of the baseline wild-type variants of the 2008 influenza season. Timeline analysis of the reported instances of HA 310-316 sequences supports this hypothesis and argues that reported in 2009 cases of (T305S+I312V+I315V) triple mutation are likely to represent tail-end of the 2008 H1N1 infection silent wave (FIG. 3C).

Initial analysis of viral sequences isolated from postmortem samples showed a high frequency of mutations in the HA 301-316 segment. In three of the first seven cases analyzed, the HA Q310H mutation was identified (see Table 2, patient id nos.: GQ414768, GQ915019, and GQ915020). Thus, in this early sample space, 3 out of 7 or 43% of individuals who died from laboratory-confirmed cases of the pandemic (H1N1) virus were infected with a virus carrying the HA Q310H mutation. In contrast, the incidence of the HA Q310H mutation overall, excluding necropsy samples, is only 4.29% (based on 47 instances of the mutation out of 1,095 viral sequences in the NCBI database as of Sep. 16, 2009). Analysis of viral isolates from additional fatal cases confirmed that the HA Q310H mutation was disproportionately high in viral infections that resulted in death. Of 65 fatal cases analyzed, 14 carried the HA Q310H mutation, or 21.5%. These 14 cases represented patients from three geographically distinct locations (Brazil, India, and Greece) further indicating that the virus underwent a rapid global evolution.

The fatality rate of patients infected with the viral variant carrying the HA Q310H mutation can be estimated at 6.38% which is significantly higher than the worldwide estimates of death rates (FIG. 3E; p=0.0016; two-tail Fisher's exact test). Notably, 7 viruses with HA mutations at the position D239, which represents both a receptor-binding residue and Ca antigenic site, were recovered from four necropsy samples (see Table 2) indicating that 86% of viral strains recovered from the postmortem samples have HA mutations.

TABLE 2
Q310H hemagglutinin mutations in virus isolates from necropsy tissues of
patients with fatal cases of the pandemic (H1N1) 2009

| GenBank | | | | |
|---|---|---|---|---|
| Accession | Collection | | | |
| Number | Date | Tissue | HA 301-316 sequence | HA 219-240 sequence |
| GQ414768 | 3 Jul. 09 | Blood | G A I N T S L P F H N I H P I T | G S S R Y S K K F K P E I A I R P K V R D Q |
| | | | (SEQ ID NO: 11) | (SEQ ID NO: 12) |
| GQ915017 | 19 Jul. 09 | Lung | G A I N T S L P F Q N I H P I T | G T S R Y S K K F K P E I A I R P K V R G Q |
| | | | (SEQ ID NO: 13) | (SEQ ID NO: 14) |
| GQ915018 | 1 Aug. 09 | Lung | G A I N T S L P F Q N I H P I T | G T S R Y S K K F K P E I A I R P K V R G Q |
| | | | (SEQ ID NO: 15) | (SEQ ID NO: 16) |
| 00915019 | 1 Aug. 09 | Lung | G A I N T S L P F H N I H P I T | G S S R Y S K K F K P E I A I R P K V R N Q |
| | | | (SEQ ID NO: 17) | (SEQ ID NO: 18) |
| 00915020 | 1 Aug. 09 | Lung | G A I N T S L P F H N I H P I T | G S S R Y S K K F K P E I A I R P K V R D Q |
| | | | (SEQ ID NO: 19) | (SEQ ID NO: 20) |
| 00915021 | 2 Aug. 09 | Trachea | G A I N T S L P F Q N I H P I T | G T S R Y S K K F K P E I A I R P K V R N Q |
| | | | (SEQ ID NO: 21) | (SEQ ID NO: 22) |
| GQ915022 | 1 Aug. 09 | Lung | G A I N T S L P F Q N I H P I T | G T S R Y S K K F K P E I A I R P K V R D Q |
| | | | (SEQ ID NO: 23) | (SEQ ID NO: 24) |

Legend: Viruses were isolated from the postmortem samples collected on indicated dates from patients with fatal cases of the (H1N1) pandemic 2009 in Brazil

### Geographical and Timeline Associations of Emergence of Novel HA Mutations and Evolution of Clinical Severity of the Pandemic (H1N1) 2009.

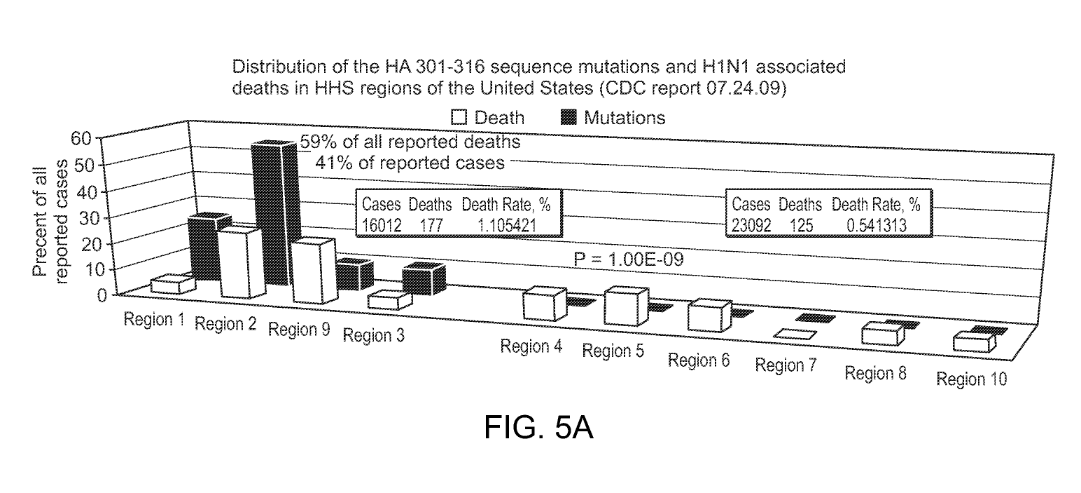

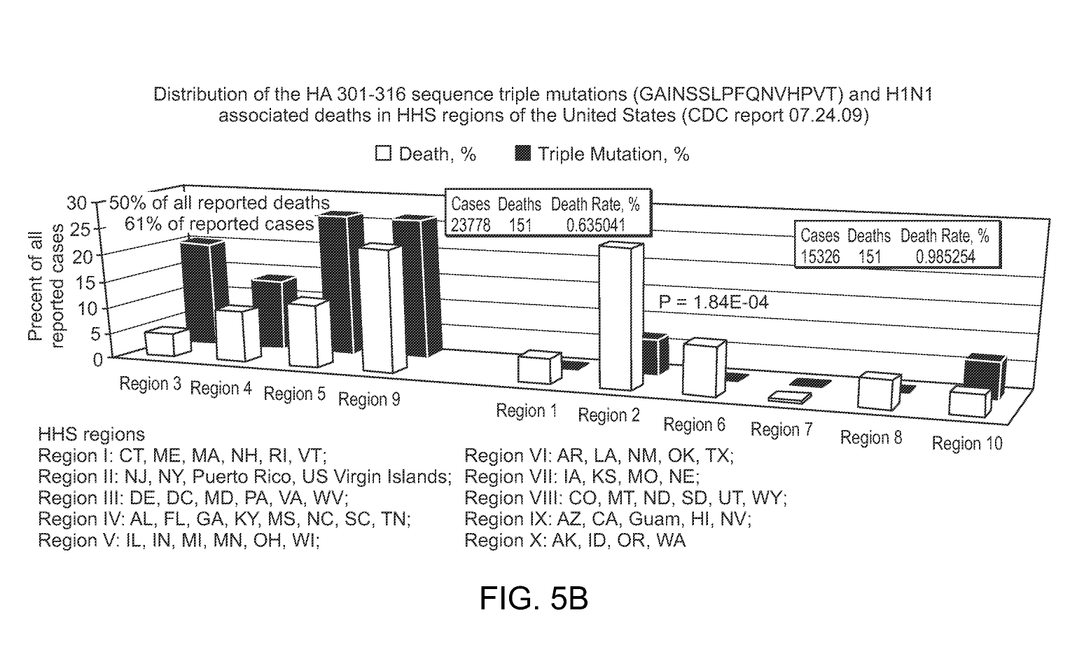

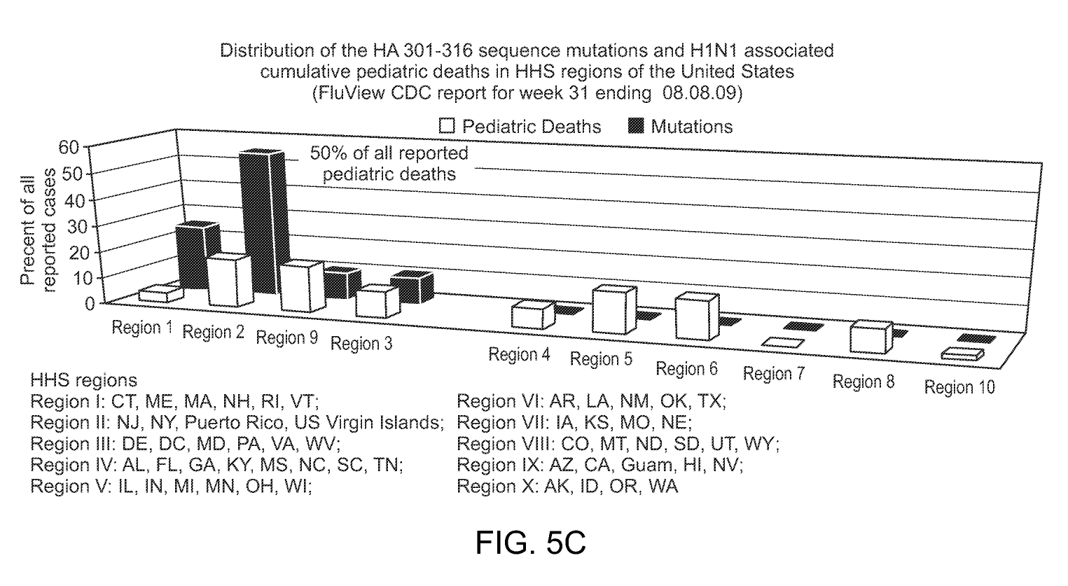

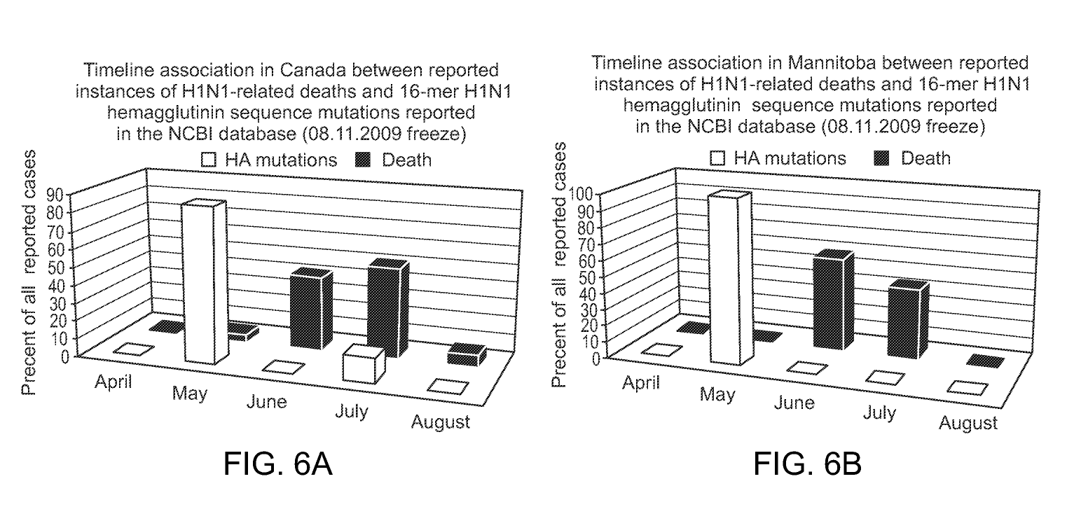

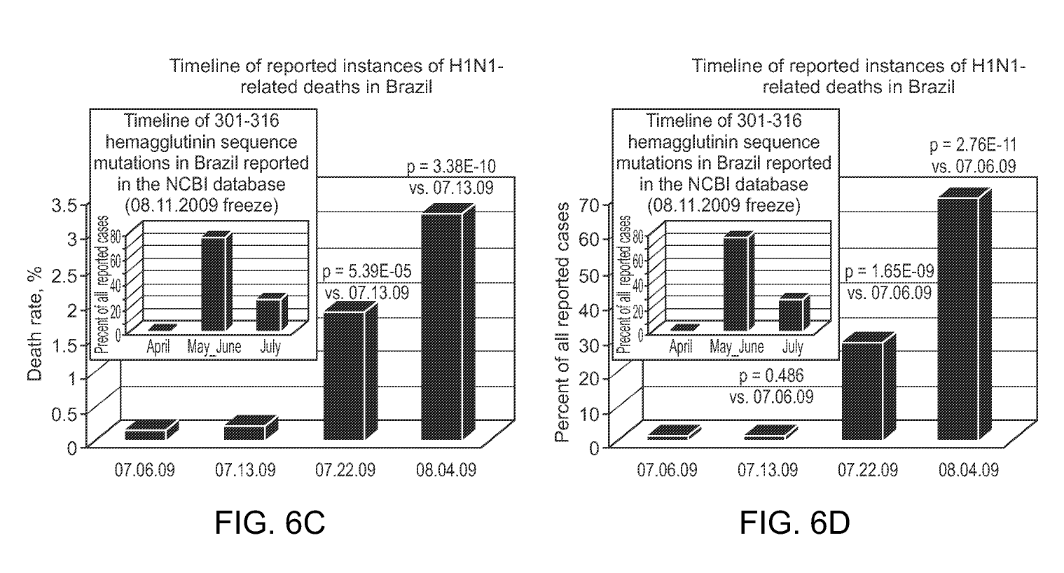

There was a statistically significant increase in the percentage of reported cases of the novel HA 301-316 mutations that correlated with the apparent increase in worldwide estimates of death rates during the pandemic (FIG. 4D-F). Analysis of timeline associations in New York (FIG. 4G), Canada, and Brazil (FIG. 6) between reported instances of H1N1-related deaths and emergence HA 301-316 sequence mutations shows that in all cases the collection of samples with subsequently detected mutations precedes the increase in H1N1-associated deaths. Analysis of the distribution of the HA 301-316 sequence mutations and reporting by the CDC H1N1-associated deaths in HHS-defined regions of the United States reinforces the conclusion that emergence of the new 2009 HA 301-316 sequence mutations is a marker of the more severe infection which is statistically more likely to cause fatal outcomes (FIG. 5). In comparison to regions with no cases of HA mutations or regions with HA triple mutation, regions with reported instances of the new 2009 HA 301-316 sequence mutations account for disproportionally large fractions of H1N1-realted deaths and have significantly higher H1N1-associated mortality rate (p=1.00E-09; 2-tailed Fisher's exact test).

### CONCLUSIONS

Our analysis indicates that pandemic (H1N1) 2009 underwent a rapid divergent global evolution that was associated with changes in clinical severity and an increase in the number of accumulated mutations in a small segment of the HA protein defined by amino acids 301 to 316. We further demonstrate the practical utility of evaluating the clinical severity of a pandemic by analyzing the case fatality ratio compared to the number of hospitalizations (“CF/H”) across different countries. Our data show that increasing CF/H ratios over time in different countries is an indicator of increasing clinical severity. Our data further show that mutations in the HA protein segment defined by amino acids 301 to 316 provides a valuable marker to identify more virulent strains of the virus which evolved during the pandemic period. Thus, the CF/H ratio, alone or in combination with identification of the marker mutations in the HA protein provides a valuable tool for identifying the emergence of a more virulent strain of a virus during a pandemic. The emergence of the marker mutations in the HA protein can also be used, alone or in combination with the CF/H ratio to identify a more virulent strain of the virus.

### Methods

We assessed the clinical severity of the pandemic (H1N1) 2009 by analyzing case fatality ratio to number of hospitalizations in United States, Canada, United Kingdom, and Australia based on official reports of public health agencies of corresponding countries. Identification of viral sample collection sources and dates, nucleotide and protein sequence analysis of the influenza hemagglutinin (HA) gene were performed using systematic survey and manual curation of each entry in the NCBI H1N1 sequence database (freeze Aug. 11, 2009). Sequence homology searches and alignments were performed using BLAST software. Assessment of the statistical validity of the findings was performed using 2-tailed Fisher's exact and Chi-square tests. All utilized software and primary data analyzed in this study are publicly available as internet-accessible resources.

### REFERENCES

1. Morens, D. M. and J. K. Taubenberger, Understanding Influenza Backward, J. Am. Med. Assoc. 2009 302(6):679-680.
2. Fraser C. et al. WHO Rapid Pandemic Assessment Collaboration. Pandemic potential of a strain of influenza A (H1N1): early findings. Science. 2009 324: 1557-61.
3. Munster V. J. et al., Pathogenesis and Transmission of Swine-Origin 2009 A(H1N1) Influenza Virus in Ferrets. Science. 2009; 325:481-3.
4. Maines T. R. et al., Transmission and Pathogenesis of Swine-Origin 2009 A(H1N1) Influenza Viruses in Ferrets and Mice. Science. 2009; 325:484-7.
5. Itoh Y., Shinya K., Kiso M., et al. In vitro and in vivo characterization of new swine-origin H1N1 influenza viruses. Nature. 2009 460:1021-5.
6. Yang Y. et al., The Transmissibility and Control of Pandemic Influenza A (H1N1) Virus. Science. 2009 Sep. 10. [Epub ahead of print]. PMID: 19745114
7. Balcan D. et al., Seasonal transmission potential and activity peaks of the new influenza A (H1N1): a Monte Carlo likelihood analysis based on human mobility. BMC Med. 2009 Sep. 10; 7(1):45. [Epub ahead of print]. PMID: 19744314
8. Reid, A. H., Fanning, T. G., Hultin, J. V. and Taubenberger, J. K. 1999. Origin and evolution of the 1918 ‘Spanish’ influenza virus hemagglutinin gene. Proc. Natl. Acad. Sci. U.S.A. 96 (4), 1651-1656.
9. Greenberg M. E., et al. Response after One Dose of a Monovalent Influenza A (H1N1) 2009 Vaccine—Preliminary Report. N Engl J. Med. 2009 Sep. 10. [Epub ahead of print]. PMID: 19745216
10. Clark T. W. et al., Trial of Influenza A (H1N1) 2009 Monovalent MF59-Adjuvanted Vaccine—Preliminary Report. N Engl J. Med. 2009 Sep. 10. [Epub ahead of print]. PMID: 19745215

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method for identifying a virulent strain of a virus, the method comprising detecting a mutation within amino acid residues 301 to 316 of the hemagglutinin protein (HA) of the virus, wherein the virus was isolated from human cells or tissues, and wherein the detection of one or more mutations indicates that a virulent strain of the virus has been identified.

2. The method of claim 1, wherein the virus is an influenza virus.

3. The method of claim 2, wherein the virus is an influenza A virus.

4. The method of claim 3, wherein the virus is of the H1 subtype.

5. The method of claim 4, wherein the mutation is with reference to the amino acid sequence G A I N T S L P F Q N I H P I T (SEQ ID NO:5).

6. The method of claim 5, wherein the mutation is selected from one of the following: a substitution of glutamine at amino acid position 310 with histidine (Q310H); the triple substitution of threonine at position 305 with serine, isoleucine at position 312 with valine, and isoleucine at position 315 with valine (T305S+I312V+I315V); a substitution of isoleucine at position 315 with valine (I315V); a substitution of proline at 314 with serine (P3145); a substitution of threonine at position 305 with serine (T305S); a double substitution of threonine at position 305 with serine and isoleucine at position 315 with valine (T305S+I315V); a substitution of histidine at 313 with tyrosine (H313Y); a substitution of alanine at 302 with serine (A302S), or one or more of the foregoing in combination.

7. The method of claim 5, wherein the mutation is selected from one of the following single amino acid substitutions: Q310H, I315V, P3145, T305S, H313Y, A302S.

8. The method of claim 7, wherein the mutation is Q310H.

9. The method of claim 6, further comprising detecting a substitution of aspartate with glutamate at position 239 (D239E) of the HA protein (an HA D239E mutation).

10. The method of claim 6, further comprising detecting a substitution of the histidine at position 274 of the neuraminidase protein (NA) with a tyrosine (an NA H274Y mutation).

11. A computer-implemented method for identifying a virulent strain of a virus, the method comprising (i) receiving a first data corresponding to a number of individuals admitted to a hospital for treatment of the virus; (ii) receiving a second data corresponding to a number of individuals who subsequently die after being admitted to the hospital for treatment of the virus; and (iii) determining the case fatality rate for hospitalization (CFR/H) using the first and second data, wherein the CFR/H is determined by dividing the number in the second data by the number in the first data, and wherein an increase in CFR/H over a period of time indicates a virulent strain of the virus.

12. The method of claim 11, wherein the first and second data are received from at least one database.

13. The method of claim 11, wherein the receiving further comprises using a processor, querying the first and second data from the at least one database.

14. The method of claim 11, wherein the CFR/H ratio is determined using first and second data obtained from multiple hospitals located in more than one geographic region.

15. The method of claim 14, wherein the more than geographic region is selected from a county, a state, a province, a country, or a continent.

16. The method of claim 11, wherein the period of time is 4 to 8 weeks, 4 to 12 weeks, or 4 to 24 weeks.

17. The method of claim 11, further comprising identifying a change in one or more of amino acids 301 to 316 of the HA protein of the virus isolated from fatal cases relative to either (i) virus isolated from nonfatal cases earlier in time or (ii) virus isolated from contemporaneous nonfatal cases, wherein a change in one or more of amino acids 301 to 316 of the HA protein of the virus isolated from fatal cases indicates a more virulent strain of the virus.

18. The method of claim 11, further comprising identifying a change in one or more of amino acids 301 to 316 of the HA protein of the virus relative to the reference sequence G A I N T S L P F Q N I H P I T (SEQ ID NO:5).

19. A method for diagnosing a virulent viral infection in a human subject, the method comprising detecting a virulent strain of a virus according to claim 1, wherein a virulent viral infection is diagnosed if a virulent strain of the virus is detected.

20. A kit comprising a set of primers for detecting a mutation in the RNA sequence of the virus encoding amino acid residues 301 to 316 of the hemagglutinin protein (HA).

21. The kit of claim 18, further comprising an antibody for detecting a mutation in amino acid residues 301 to 316 of the hemagglutinin protein (HA) of the virus.

22. A system for performing the method of claim 11 comprising: a memory; a processor coupled to a memory and configured to receive a first data corresponding to a number of individuals admitted to a hospital for treatment of the virus; receive a second data corresponding to a number of individuals who subsequently die after being admitted to the hospital for treatment of the virus.

23. The system according to claim 22, wherein the processor is configured to query the first and second data from at least one database.

24. A computer program product comprising a machine-readable medium storing instructions that, when executed by at least one processor, cause the at least one processor to perform operations comprising (i) receiving a first data corresponding to a number of individuals admitted to a hospital for treatment of the virus; (ii) receiving a second data corresponding to a number of individuals who subsequently die after being admitted to the hospital for treatment of the virus; and (iii) determining the case fatality rate for hospitalization (CFR/H) using the first and second data according to the method of claim 11.

25. The computer program product according to claim 24, wherein the receiving operations further comprise using a processor, querying the first and second data from the at least one database.


SEQUENCE LISTING (25 sequences)

SEQ ID NO: 1 - protein, 16 aa, Influenza virus
GAINTSLPFHNIHPIT

SEQ ID NO: 2 - protein, 16 aa, Influenza virus
GAINSSLPFQNVHPVT

SEQ ID NO: 3 - protein, 16 aa, Influenza virus
GAINTSLPFQNIHPVT

SEQ ID NO: 4 - protein, 16 aa, Influenza virus
GAINTSLPFQNIHSIT

SEQ ID NO: 5 - protein, 16 aa, Influenza virus
GAINTSLPFQNIHPIT

SEQ ID NO: 6 - protein, 16 aa, Influenza virus
GAINSSLPFQNIHPVT

SEQ ID NO: 7 - protein, 16 aa, Influenza virus
  misc_feature (10)..(10): Xaa can be any naturally occurring amino acid
GAINTSLPFXNIHPIT

SEQ ID NO: 8 - protein, 16 aa, Influenza virus
GAINTSLPFQNIYPIT

SEQ ID NO: 9 - protein, 16 aa, Influenza virus
GSINTSLPFQNIHPIT

SEQ ID NO: 10 - protein, 16 aa, Influenza virus
  misc_feature (2)..(2): Xaa can be any naturally occurring amino acid
GXINTSLPFQNIHPIT

SEQ ID NO: 11 - protein, 16 aa, Influenza virus
GAINTSLPFHNIHPIT

SEQ ID NO: 12 - protein, 22 aa, Influenza virus
GSSRYSKKFKPEIAIRPKVRDQ

SEQ ID NO: 13 - protein, 16 aa, Influenza virus
GAINTSLPFQNIHPIT

SEQ ID NO: 14 - protein, 22 aa, Influenza virus
GTSRYSKKFKPEIAIRPKVRGQ

SEQ ID NO: 15 - protein, 16 aa, Influenza virus
GAINTSLPFQNIHPIT

SEQ ID NO: 16 - protein, 22 aa, Influenza virus
GTSRYSKKFKPEIAIRPKVRGQ

SEQ ID NO: 17 - protein, 16 aa, Influenza virus
GAINTSLPFHNIHPIT

SEQ ID NO: 18 - protein, 22 aa, Influenza virus
GSSRYSKKFKPEIAIRPKVRNQ

SEQ ID NO: 19 - protein, 16 aa, Influenza virus
GAINTSLPFHNIHPIT

SEQ ID NO: 20 - protein, 22 aa, Influenza virus
GSSRYSKKFKPEIAIRPKVRDQ

SEQ ID NO: 21 - protein, 16 aa, Influenza virus
GAINTSLPFQNIHPIT

SEQ ID NO: 22 - protein, 22 aa, Influenza virus
GTSRYSKKFKPEIAIRPKVRNQ

SEQ ID NO: 23 - protein, 16 aa, Influenza virus
GAINTSLPFQNIHPIT

SEQ ID NO: 24 - protein, 22 aa, Influenza virus
GTSRYSKKFKPEIAIRPKVRDQ

SEQ ID NO: 25 - protein, 566 aa, Influenza virus
MEARLLVLLCAFAATNADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHHQNEQGSGYAADQKSTQNAIDGITNKVNSVIEKMNTQFTAVGKEFNNLERRIENLNKKVDDGFLDIWTYNAELLVLLENERTLDFHDSNVRNLYEKVKSQLKNNAKEIGNGCFEFYHKCDDACMESVRNGTYDYPKYSEESKLNREEIDGVKLESMGVYQILAIYSTVASSLVLLVSLGAISFWMCSNGSLQCRICI